(19) United States
(12) Patent Application Publication
(10) Pub. No.: US 2014/0257501 A1
(43) Pub. Date: Sep. 11, 2014

(54) Joint Energy Absorbing System and Method of Use

(71) Applicant: MOXIMED, INC., Hayward, CA (US)

(72) Inventors: David Lowe, Redwood City, CA (US); Anton G. Clifford, Mountain View, CA (US); Mary O'Connell, Menlo Park, CA (US); Stefan Gabriel, Mattapoisett, MA (US); Heber Saravia, San Francisco, CA (US); Ezra T. Schiff, Mountain View, CA (US)

(73) Assignee: MOXIMED, INC., Hayward, CA (US)

(21) Appl. No.: 14/200,552

(22) Filed: Mar. 7, 2014

Related U.S. Application Data
(60) Provisional application No. 61/775,365, filed on Mar. 8, 2013.

(51) Int. Cl. A61F 2/38; A61B 17/17

(52) U.S. Cl. CPC A61F 2/3836; A61B 17/1764
USPC 623/20.22; 623/20.21; 606/96

(57) ABSTRACT

An energy absorbing system, useful for attachment across a patient's joint, includes portions which are formed entirely or in part of a non-metallic material, e.g., PAEK, and thus reduces the possibility of the formation of metal debris in vivo. The system can include a flexibility-enhanced piston, retained within a spring, which reduces the possibility of tissue impingement with the system.

### BACKGROUND

1. Field of Endeavor

The present invention relates to devices, systems, and processes useful to absorb energy, and more specifically in application across a load-bearing joint of a patient.

2. Brief Description of the Related Art

Joint replacement is one of the most common and successful operations in modern orthopaedic surgery. It consists of replacing painful, arthritic, worn or diseased parts of a joint with artificial surfaces shaped in such a way as to allow joint movement. Osteoarthritis is a common diagnosis leading to joint replacement. Such procedures are a last resort treatment as they are highly invasive and require substantial periods of recovery. Total joint replacement, also known as total joint arthroplasty, is a procedure in which all articular surfaces at a joint are replaced. This contrasts with hemiarthroplasty (half arthroplasty) in which only one bone's articular surface at a joint is replaced and unincompartmental arthroplasty in which the articular surfaces of only one of multiple compartments at a joint (such as the surfaces of the thigh and shin bones on just the inner side or just the outer side at the knee) are replaced. Arthroplasty, as a general term, is an orthopaedic procedure which surgically alters the natural joint in some way. This includes procedures in which the arthritic or dysfunctional joint surface is replaced with something else, procedures which are undertaken to reshape or realign the joint by osteotomy or some other procedure. As with joint replacement, these other arthroplasty procedures are also characterized by relatively long recovery times and are highly invasive procedures. A previously popular form of arthroplasty was interpositional arthroplasty in which the joint was surgically altered by insertion of some other tissue like skin, muscle or tendon within the articular space to keep inflammatory surfaces apart. Another previously done arthroplasty was excisional arthroplasty in which articular surfaces were removed leaving scar tissue to fill in the gap. Among other types of arthroplasty are resection(al) arthroplasty, resurfacing arthroplasty, mold arthroplasty, cup arthroplasty, silicone replacement arthroplasty, and osteotomy to affect joint alignment or restore or modify joint congruity. When it is successful, arthroplasty results in new joint surfaces which serve the same function in the joint as did the surfaces that were removed. Any chondrocytes (cells that control the creation and maintenance of articular joint surfaces), however, are either removed as part of the arthroplasty, or left to contend with the resulting joint anatomy. Because of this, none of these currently available therapies are chondro-protective.

A widely-applied type of osteotomy is one in which bones are surgically cut to improve alignment. A misalignment due to injury or disease in a joint relative to the direction of load can result in an imbalance of forces and pain in the affected joint. The goal of osteotomy is to surgically realign the bones at a joint and thereby relieve pain by equalizing forces across the joint. This can also increase the lifespan of the joint. When addressing osteoarthritis in the knee joint, this procedure involves surgical realignment of the joint by cutting and reattaching part of one of the bones at the knee to change the joint alignment, and this procedure is often used in younger, more active or heavier patients. Most often, high tibial osteotomy (HTO) (the surgical realignment of the upper end of the shin bone (tibia) to address knee malalignment) is the osteotomy procedure done to address osteoarthritis and it often results in a decrease in pain and improved function. However, HTO addresses only mechanical alignment and not ligamentous instability. HTO is associated with good early results, but results deteriorate over time.

Other approaches to treating osteoarthritis involve an analysis of loads which exist at a joint. Both cartilage and bone are living tissues that respond and adapt to the loads they experience. Within a nominal range of loading, bone and cartilage remain healthy and viable. If the load falls below the nominal range for extended periods of time, bone and cartilage can become softer and weaker (atrophy). If the load rises above the nominal level for extended periods of time, bone can become stiffer and stronger (hypertrophy). Finally, if the load rises too high, then abrupt failure of bone, cartilage and other tissues can result. Accordingly, it has been concluded that the treatment of osteoarthritis and other bone and cartilage conditions is severely hampered when a surgeon is not able to precisely control and prescribe the levels of joint load. Furthermore, bone healing research has shown that some mechanical stimulation can enhance the healing response and it is likely that the optimum regime for a cartilage/bone graft or construct will involve different levels of load over time, e.g., during a particular treatment schedule. Thus, there is a need for devices which facilitate the control of load on a joint undergoing treatment or therapy, to thereby enable use of the joint within a healthy loading zone.

Certain other approaches to treating osteoarthritis contemplate external devices such as braces or fixators which attempt to control the motion of the bones at a joint or apply cross-loads at a joint to shift load from one side of the joint to the other. A number of these approaches have had some success in alleviating pain but have ultimately been unsuccessful due to lack of patient compliance or the inability of the devices to facilitate and support the natural motion and function of the diseased joint. The loads acting at any given joint and the motions of the bones at that joint are unique to the joint and to the person. For this reason, any proposed treatment based on those loads and motions must account for this variability to be universally successful. The mechanical approaches to treating osteoarthritis have not taken this into account and have consequently had limited success.

Prior approaches to treating osteoarthritis have also failed to account for all of the basic functions of the various structures of a joint in combination with its unique movement. In addition to addressing the loads and motions at a joint, an ultimately successful approach must also acknowledge the dampening and energy absorption functions of the anatomy, and be implantable via a minimally invasive technique. Prior devices designed to reduce the load transferred by the natural joint typically incorporate relatively rigid constructs that are incompressible. Mechanical energy (E) is the action of a force (F) through a distance (s) (i.e., E=F×s). Device constructs which are relatively rigid do not allow substantial energy storage as the forces acting on them do not produce substantial deformations—do not act through substantial distances—within them. For these relatively rigid constructs, energy is transferred rather than stored or absorbed relative to a joint. By contrast, the natural joint is a construct comprised of elements of different compliance characteristics such as bone, cartilage, synovial fluid, muscles, tendons, ligaments, etc. as described above. These dynamic elements include relatively compliant ones (ligaments, tendons, fluid, cartilage) which allow for substantial energy absorption and storage, and relatively stiffer ones (bone) that allow for efficient energy transfer. The cartilage in a joint compresses under applied force and the resultant force displacement product represents the energy absorbed by cartilage. The fluid content of cartilage also acts to stiffen its response to load applied quickly and dampen its response to loads applied slowly. In this way, cartilage acts to absorb and store, as well as to dissipate energy.

With the foregoing applications in mind, it has been found to be necessary to develop effective structures for mounting to body anatomy. Such structures should conform to body anatomy and cooperate with body anatomy to achieve desired load reduction, energy absorption, energy storage, and energy transfer. These structures should include mounting means for attachment of complementary structures across articulating joints.

Currently, an energy absorbing system developed by Moximed is implanted in two pieces. In that procedure: the surgeon gains access to the medial knee from the contralateral side; two 5-8 cm incisions are made, connected by a skin bridge and a subcutaneous tunnel; a femoral target, i.e., location for mounting the femoral base, is selected; the absorber alignment relative to the joint, is determined; a femoral base is selected and implanted; the energy absorber and the tibial base are connected ex-vivo; the absorber is inserted into the tissue tunnel; the absorber is connected to the femoral base; the tibial base is aligned and fixed; and the absorber is activated by releasing a restraining device.

For these implant structures to function optimally, they must not cause an adverse disturbance to joint motion. Therefore, what is needed is an approach which addresses both joint movement and varying loads as well as complements underlying or adjacent anatomy.

### SUMMARY

According to a first aspect of the invention, an implantable energy absorbing system useful for absorbing energy from a joint of a patient comprises a proximal base configured and arranged for implantation adjacent to said joint, a distal base configured and arranged for implantation adjacent to said joint on a side of said joint opposite said proximal base, and an energy absorbing device attached to both the proximal base and the distal base, the energy absorbing device comprising a spring having a hollow interior, and a piston at least partially located in said spring hollow interior, wherein the spring is a metallic spring and the piston is formed of a non-metallic material.

At least one of the spring and the piston can be formed of PAEK.

The energy absorbing device and the two bases can comprise two ball-and-socket connectors connecting together each base with the energy absorbing device.

The energy absorbing device can include proximal and distal ends, and each of said ball-and-socket connectors can comprise a socket on one of said bases and a ball on each of said absorbing device proximal and distal ends, each of said balls being rotatably received in one of said sockets.

At least one base can include a removable socket connector and a mating structure on said base, the mating structure being configured and arranged to receive said removable socket connector, the removable socket connector can include a socket body, a socket formed in said socket body configured to receive said ball, and a lumen extending from said socket, and the piston can comprise a ball and is sized so that said piston can be pushed through said lumen until said ball is captured in said socket.

At least one of the bases can comprise a lumen having two ends, said socket being located at one of said base lumen ends, and said piston can comprise a ball and is sized so that said piston can be pushed through said lumen until said ball is captured in said socket.

Such a system can further comprise a plug sized to at least partially fill said base lumen.

The base can comprise a socket body split in two portions, each of said portions including a mating portion which permits the two socket body portions to be removably connected together.

The spring can comprise a helical spring.

The spring hollow interior has a length, and said piston can extend within said spring hollow interior a distance less than said length.

The spring is not secured to opposite ends of the energy absorbing device and is floating on the piston.

The energy absorbing device can further comprise two ends, the piston and spring extending toward each other in opposite directions from each of said ends, and a piston guide tube extending within said spring and around said piston.

The energy absorbing device can further comprise two ends, the piston and spring extending toward each other in opposite directions from each of said ends, and a tubular sheath positioned on the outside of at least part of the spring, the sheath being connected to one of said two end.

The piston can comprise lateral cutouts.

The piston also comprise a base end adjacent one of said bases, a free end opposite said base end, and a blind bore extending along said piston from said free end.

The piston can also further comprise at least one lateral cutout extending between said blind bore and the exterior of said piston.

At least one lateral cutout can comprise at least one part-circumferential cutout.

At least one lateral cutout can comprises a plurality of part-circumferential cutouts spaced along said piston between said ends.

The piston can comprise a frustoconical taper along its length.

The piston can comprise at least one groove extending in a direction between said ends.

The piston can also comprise a base end adjacent one of said bases, a free end opposite said base end, wherein said free end comprises a metal tip, and wherein portions of said piston between said metal tip and said base end are formed of PAEK.

The piston can further also comprise a base end adjacent one of said bases, a free end opposite said base end, and a telescoping piston extension positioned around said free end.

The telescoping piston extension can include a blind bore having an inner diameter, and said piston free end has an outer diameter less than said blind bore inner diameter such that said telescoping piston extension can slide along said piston free end.

The telescoping piston extension can also include a lip on said blind bore, and said piston includes a lip adjacent said free end sized to catch on said telescoping piston extension lip and prevent said telescoping piston extension from sliding off said piston free end.

The piston can comprise at least one flattened exterior portion.

At least one of the bases can comprise a top surface and a periosteum-contacting surface, at least one screw hole extending through said base between said top surface and said bone-contacting surface, and at least one periosteum-contacting ring on and extending outward from said periosteum-contacting surface, adjacent to and surrounding said at least one screw hole.

At least one of the bases can also comprise a top surface and a periosteum-contacting surface, and at least one standoff extending from said periosteum-contacting surface.

At least one of said bases can comprise slots and/or lumens formed through the base.

Another aspect includes an implantable energy absorbing system useful for absorbing energy from a joint of a patient, the system comprising a proximal base configured and arranged for implantation adjacent to said joint, a distal base configured and arranged for implantation adjacent to said joint on a side of said joint opposite said proximal base, and an energy absorbing device attached to both the proximal base and the distal base, the energy absorbing device comprising a spring having a hollow interior, and a piston at least partially located in said spring hollow interior, wherein the energy absorbing device and the two bases comprise two ball-and-socket connectors connecting together each base with the energy absorbing device, at least one of the ball and socket is non-metallic.

Yet another aspect includes a base useful for implantation adjacent to a joint, the base comprising a top surface and a periosteum-contacting surface, at least one screw hole extending through said base between said top surface and said bone-contacting surface, and at least one periosteum-contacting ring on and extending outward from said periosteum-contacting surface, adjacent to and surrounding said at least one screw hole.

Another aspect includes an energy absorber spacer comprising a trough-shaped portion having two sidewalls, a bottom wall connected to each of the sidewalls, and an open top, and a handle extending from said trough-shaped portion and away from said bottom wall.

The bottom wall and said two sidewalls can define a trough with open ends.

Yet another aspect includes a tibial alignment guide comprising a base having base body and a pin receiver on said base body including a through lumen, and a post having a through lumen and first and second ends, and an indicator arm, wherein the post and the indicator arm comprise complementary mating structures which permit the indicator arm to be mounted adjacent to said post first end extending away from the post at a single fixed angular orientation, and wherein said post second end and said base together comprise a removable connector configured and arranged to permit the post to be connected to the base at a single, fixed angular orientation.

The removable connector can comprise an enlarged bearing and a protrusion extending laterally from said bearing, a toroidal receiver including an interior surface sized and configured to receive said enlarged bearing, and a tapered slot in said toroidal receiver sized to receive said protrusion, such that when said enlarged bearing is positioned in said toroidal receiver with said protrusion located in said tapered slot, said indicator arm is oriented in a single fixed angular orientation relative to said post lumen and said base pin receiver.

A further aspect relates to a method of implanting an energy absorbing system into a patient having a joint with a joint line, the method comprising forming a femoral incision that begins slightly distal to a midpoint of Blumensaat's line and extends away from the joint line, forming a tibial incision that begins at a posterior tibial K-wire hole and extends through the location of the distal tibial K-wire hole, forming a tissue tunnel from the tibial incision to the femoral incision, aligning a tibial positioning guide with a femoral trial indicator to correctly align the tibia and femur, inserting a K-wire through a hole in a distal portion of the tibial positioning guide, positioning two femoral and two tibial K-wires in the femur and tibia, respectively, removing the femoral trial and the tibial positioning guide, and inserting a femoral end of an energy absorbing system, including a femoral base, an absorber, and a tibial base, connected together, through the tibial incision and bringing the femoral base through the tunnel posterior to the femoral K-wires.

Such a method can further comprise engaging the femoral Base with the two femoral K-wires and stabilizing the femoral base by inserting a K-wire through a hole in the femoral base.

Such a method can further comprise setting a minimum spacing between the absorber and the patient's tissues, including introducing an absorber spacer through the femoral incision and beneath the absorber.

Such a method can further comprise positioning the absorber parallel to the tibial shaft.

Such a method can further comprise mounting the femoral base to the femur, and mounting the tibial base to the tibia.

Such a method can further comprise activating the absorber.

Yet a further aspect includes a method of implanting an energy absorbing system in a patient, the method comprising inserting, as a single interconnected unit, a femoral base, a tibial base, and an absorber rotatably connected to the femoral and tibial bases, positioning the femoral base on the distal end of a femur, positioning the tibial base on the proximal end of the tibia, positioning the absorber across the knee joint to absorb load ordinarily carried by the knee joint, and securing the femoral and tibial bases to the bone with bone screws.

The inserting step can include inserting through a tissue tunnel from an incision adjacent the tibia.

The step of positioning the absorber can include positioning the absorber substantially parallel to the tibial axis when the knee joint is at full extension.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates a top plan view of an exemplary energy absorbing system;

FIG. 2 illustrates a top plan view of a first embodiment of an energy absorbing device;

FIG. 3 illustrates a cross-sectional view of the embodiment of FIG. 2;

FIG. 4 illustrates a top plan view of a second embodiment of an energy absorbing device;

FIG. 5 illustrates a cross-sectional view of the embodiment of FIG. 4;

FIG. 6 illustrates a top plan view of a third embodiment of an energy absorbing device;

FIG. 7 illustrates a cross-sectional view of the embodiment of FIG. 6;

FIG. 8 illustrates a perspective view of a fourth embodiment of an energy absorbing device;

FIG. 9 illustrates a perspective, cross-sectional view of the embodiment of FIG. 8;

FIG. 10 illustrates a perspective view of an exemplary embodiment of a piston with enhanced transverse flexibility;

FIG. 11 illustrates a perspective view of another exemplary embodiment of a piston with enhanced transverse flexibility;

FIG. 12 illustrates a perspective view of a third exemplary embodiment of a piston with enhanced transverse flexibility;

FIG. 13 illustrates a perspective view of a fourth exemplary embodiment of a piston with reduced sliding friction;

FIG. 14 illustrates a perspective view of a fifth exemplary embodiment of a piston with reduced sliding friction;

FIG. 15 illustrates a perspective view of a sixth exemplary embodiment of a piston with reduced sliding friction;

FIG. 16 illustrates a perspective view of a seventh exemplary embodiment of a piston with reduced sliding friction;

FIG. 17 illustrates a perspective, cross-sectional view of the embodiment of FIG. 16;

FIG. 18 illustrates a perspective view of an eighth exemplary embodiment of a piston with reduced sliding friction;

FIG. 19 illustrates a perspective, cross-sectional view of the embodiment of FIG. 18;

FIG. 20 illustrates a perspective view of an ninth exemplary embodiment of a piston;

FIG. 21 illustrates a perspective, cross-sectional view of the embodiment of FIG. 20;

FIG. 22 illustrates a perspective view of an tenth exemplary embodiment of a piston;

FIGS. 23-26 illustrate top plan and a bottom perspective views of an exemplary femoral base;

FIGS. 27 and 28 illustrate top plan and a bottom perspective views of another exemplary femoral base;

FIGS. 29 and 30 illustrate top plan and a bottom perspective views of a yet another exemplary base;

FIG. 31 illustrates an exploded view of another exemplary energy absorbing system;

FIGS. 32A-32C illustrate views of the assembly of portions of yet another energy absorbing system;

FIGS. 33A-33C illustrate views of the assembly of portions of yet another energy absorbing system;

FIG. 34A illustrates a top plan view of a further exemplary energy absorbing system;

FIG. 34B illustrates a view taken at line B-B in FIG. 34A;

FIG. 34C illustrates a view taken at line C-C in FIG. 34A;

FIG. 35 illustrates a perspective view of an exemplary absorber spacer;

FIGS. 36A and 36B illustrate perspective views of a tibial alignment guide in partially-assembled and fully-assembled configurations, respectively; and

FIG. 37 illustrates steps of an exemplary implantation method.

### DETAILED DESCRIPTION OF EXEMPLARY EMBODIMENTS

Referring now to the drawing figures, like reference numerals designate identical or corresponding elements throughout the several figures. The drawings, which are provided by way of example and not limitation, illustrate disclosed embodiments which are directed towards apparatus and methods for treating the knee joint. However, these embodiments may also be used in treating other body joints, and to alleviate pain associated with the function of diseased or misaligned members forming a body joint without limiting the range of motion of the joint. The embodiments described below relate to apparatuses and methods for adjusting the amount of load an energy absorbing device can manipulate. Some embodiments include an energy absorbing device including the use of a single spring member, however other number of spring members may also be used.

Certain of the embodiments include energy absorbing devices designed to minimize and complement the dampening effect and energy absorption provided by the anatomy of the body, such as that found at a body joint. It has been postulated that to minimize pain, load manipulation or absorption of 1-40% of forces, in varying degrees, may be necessary. Variable load manipulation or energy absorption in the range of 5-20% can be a target for certain applications.

In body anatomy incorporating energy absorbing systems as described below, less forces are transferred to the bones and cartilage of the members defining the joint, and a degree of the forces between body members is absorbed by the energy absorbing system. In one embodiment, the energy absorbing system can be initially configured to eliminate, variably reduce or manipulate loads to a desired degree, and to be later adjusted or altered as patient needs are better determined or change.

In applications to the knee joint, the energy absorbing system can be designed to absorb medial compartment loads in a manner that completely preserves the articulating joint and capsular structures. One embodiment of the present invention is a load bypassing knee support system comprised of a kinematic load absorber, two contoured base components and a set of bone screws. The implanted system is both extra articular and extra capsular and resides in the subcutaneous tissue on the medial (or lateral) aspect of the knee. The device is inserted through two small incisions above the medial femoral and tibial condyles. The base components are fixed to the medial cortices of the femur and tibia using bone screws. The energy absorber having a spring value of about twenty to thirty pounds can provide therapeutic benefit for patients of 275 pounds or less. Higher spring forces would provide greater reduction in joint load and may correlate to greater symptom (i.e., pain) relief.

It has been recognized that knee forces have multiple components. There are a quadriceps force F_{Q} and a ground reaction force F_{G} directed generally longitudinally along a leg and there are lateral compartment forces F_{L} and medial compartment forces F_{M}. There is, however, no conventional clinical measure of F_{M} or F_{L}. On the other hand, there are non-axial knee forces which result in a moment being applied across the joint referred to as a knee adduction moment. The knee adduction moment (KAM) can be measured clinically. The measurements are useful as KAM can be considered to be a clinical surrogate measure for knee forces.

It has been further observed that a high knee adduction moment correlates with pain. That is, it would be expected that a group of people with diseased joints having lower KAM may not have pain whereas individuals with a relatively higher KAM would experience pain. Thus, an active reduction of knee adduction moment can reduce pain. The system of the present invention reduces the KAM of the patient.

It has also been found that a medial compartment of a knee of an average person with osteoarthritis can benefit from an absorber set for compression between 1 mm and 10 mm, and preferably 3-6 mm with a spring or absorber element that accommodates a range from 20-60 pounds. In a preferred embodiment, the absorber is set for about 4 mm of such compression and a pre-determined load of about 30 pounds.

In each of the disclosed embodiments, various features can be incorporated from other of the disclosed embodiments. Moreover, each of the contemplated embodiments can include springs formed to provide desirable energy absorbing which varies as the spring is compressed during various degrees of flexion and extension of joint markers to which the energy absorbing device is attached. The term “spring” is used throughout the description but it is contemplated to include other energy absorbing and compliant structures can be used to accomplish the functions of the invention as described in more detail below.

In certain situations, it has been found to be a benefit to implant the energy absorbing device in an inactivated condition, only later taking steps, perhaps several weeks later, to place the device into an activated state. In this way, the device can become further affixed to bone as the bone and surrounding tissue grows over portions of the device. Accordingly, each of the disclosed embodiments can be so implanted and later activated and adjusted through a patient's skin.

Further, various approaches to adjusting the energy absorbing device are contemplated and disclosed below. That is, various approaches to adjusting structure between piston and arbor structure as well as adjusting mounts to which the piston and arbor structures are configured to engage are disclosed. In the former regard, adjustable collars and adjustable link ends are contemplated approaches. Additionally, any of a variety of approaches to achieving adjustment through a patient's skin or through an incision, either through direct engagement with the energy absorbing device with a tool or by applying forces to the device through the surface of the skin, can be incorporated to fill a perceived need.

The singular forms “a,” “an,” and “the” include plural referents unless the context clearly dictates otherwise. Thus, for example, reference to “a hole” includes reference to one or more of such holes, and reference to “the shaft” includes reference to one or more of such shafts.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

For example, a range of 1 to 5 should be interpreted to include not only the explicitly recited limits of 1 and 5, but also to include individual values such as 2, 2.7, 3.6, 4.2, and sub-ranges such as 1-2.5, 1.8-3.2, 2.6-4.9, etc. This interpretation should apply regardless of the breadth of the range or the characteristic being described, and also applies to open-ended ranges reciting only one end point, such as “greater than 25,” or “less than 10.”

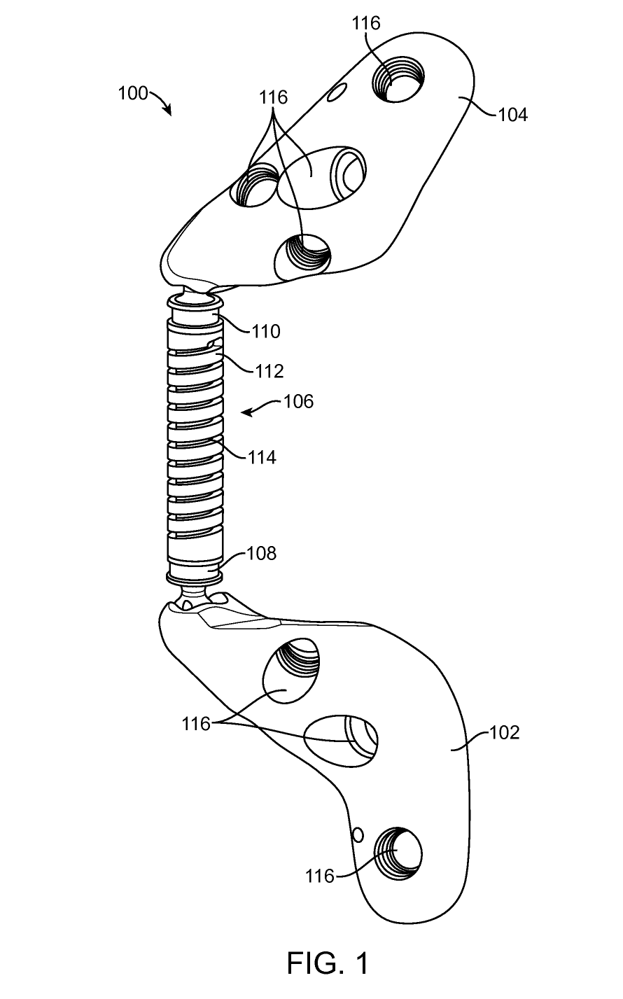

Turning now to the drawing figures, several exemplary embodiments are illustrated. FIG. 1 illustrates an energy absorbing system 100 according to a first exemplary embodiment. The system 100 includes a distal base component 102 and a proximal base component 104, which are similar in some respects to those described in U.S. patent application Ser. No. 13/488,102, assigned to Moximed, and to those described in U.S. Pat. No. 8,123,805 (“'805 patent”), the entireties of which are incorporated by reference herein. The system includes an energy absorbing device 106 attached to and mounted between the bases 102, 104, in a manner similar to that described in the '805 patent.

While prior devices have been formed of biocompatible metals, the inventors herein have determined that other materials which are biocompatible, have high wear resistance, are durable, non-interfering with MRI scanning, and are capable of being manufactured into complex geometries, are advantageously used to form some or all of the subcomponents of an energy absorbing system as described herein. Examples of materials include, but are not limited to: PAEK, PAEK composites, polycarbonate urethanes (PCU), PCU composites, ceramics, and pyrocarbon. PAEK (poly aryl ether ketone), e.g., PEEK (poly ether ether ketone), is a significantly advantageous material out of which to form some or all of the subcomponents of an energy absorbing system described herein. More specifically, each of the bases, which connect the system to a patient's bone, and the energy absorbing device there between, can perform significantly better than when those structures are formed of metals, for a number of reasons. Without being limited to a particular justification, when formed of PEEK, structures which move relative to each other (i.e., PEEK-on-PEEK articulation), for example ball-and-socket joints, do not pose the risk of the generation of metal particles when in vivo, that prior devices pose. Furthermore, PAEK is MRI compatible, which metal subcomponents generally are not, yet are radiographically invisible. According to an exemplary, yet further advantageous embodiment, CFR (carbon fiber reinforced) PEEK is used for some or all of the subcomponents described herein, with preferably 30% fiber, although other amounts and methods of reinforcement are also contemplated for portions of the device which are to be more rigid. Fibers used for reinforcement may include continuous fibers, chopped or milled fibers or other types of reinforcement. Portions, such as the spring, which are to flex in use, are advantageously formed of neat (unfilled) PEEK. Although PEEK or CFR PEEK are described as preferred materials for the subcomponents of the energy absorbing system other non-metallic materials including ceramics, biocompatible polymers and filled polymer materials can also achieve many of the advantages described herein and are useable in the energy absorbing systems.

Yet another advantageous aspect of devices described herein includes that the pistons can be formed to include flexibility enhancements, which permit the energy absorbing device to have improved lateral or transverse flexibility relative to prior devices, which assists in avoiding tissue impingement when implanted.

The energy absorbing device 106 includes a spring base or arbor 108, a piston base 110, a spring connected to the arbor 108, and a piston 114 connected to the piston base 110 and inside of the spring 112. As illustrated in FIG. 1, both of the bases 102, 104 include a plurality of screw holes 116 through the bases, which are configured to receive a bone screw or similar anchor to secure each base, and thus the system 100, to a patient's bones, preferably those on opposing sides of a joint, e.g., the femur and tibia. While the embodiments illustrated herein show pistons and springs extending in distal and proximal directions, respectively, they can be reversed.

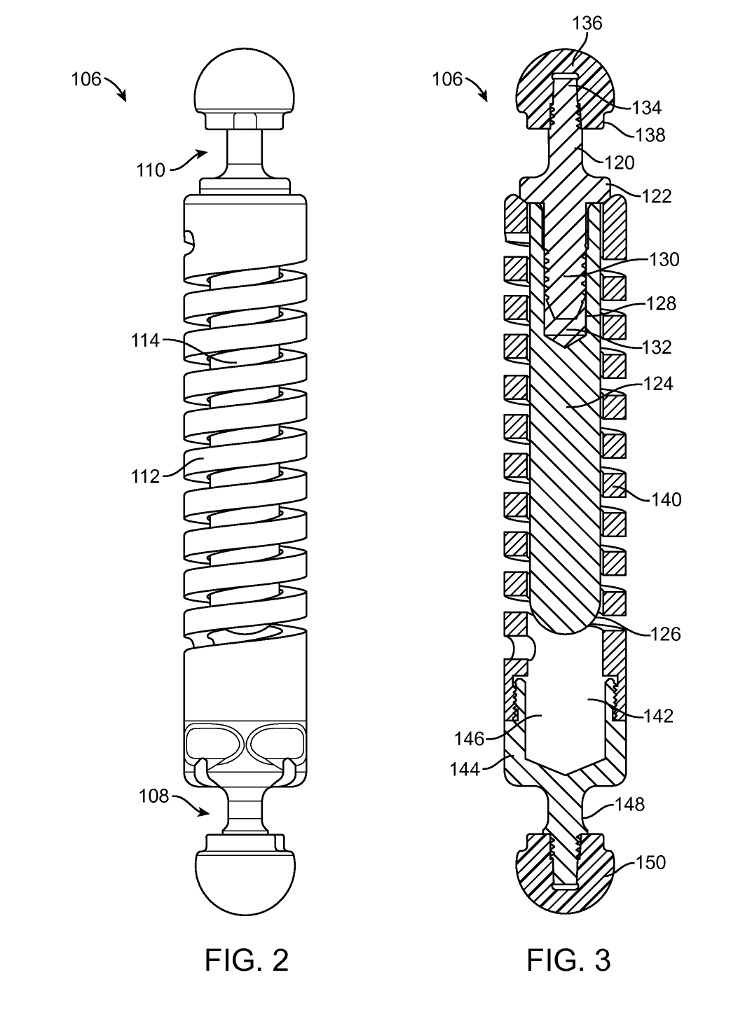

FIGS. 2 and 3 together illustrate a first exemplary embodiment 106 of an energy absorbing device embodying principles of the present invention. FIG. 2 illustrates an elevational view of the device 106, including the arbor 108, to which the spring 112 is attached, and the base 110, to which the piston 114, which rides inside the hollow interior of the spring, is attached. As illustrated in the drawing figures, the spring exterior is advantageously cylindrical, although it can be formed with other external shapes. The piston 114 and the interior shape of the spring are also advantageously cylindrical, although other complementary shapes, including ellipses, rectangles (including squares), and other, less regular shapes can alternatively be used.

With more detailed reference to FIG. 3, the piston base 110 includes a shaft 120 having an enlargement or shoulder 122 along its length which extends outwardly from the shaft. The shoulder 122 has a larger radial extent than the inner diameter of the spring 112, so that the proximal end of the spring cannot move past the shoulder. A piston body 124 is connected or otherwise mounted to the shaft 120, and includes a distal end 126, preferably rounded, opposite the shaft 120. A recess or blind bore 128 is formed at the proximal end of the piston body 124 (opposite the end 126) of a size suitable to receive therein a distal portion 130 of the shaft 120. The distal portion 130 of the shaft 120 is attached to the piston body 124 at the recess 128 with any suitable media 132, e.g., cement, a threaded connection, a weld, a braze, or the like.

Each end of the energy absorbing device 106 includes a structure which is configured to mate with corresponding structures on each base 102, 104, and permit relative rotational motion between them, while restricting or preventing the mating structures from becoming disassembled. In the exemplary embodiment of FIGS. 2 and 3, a ball connector 136 is mounted to the proximal end 134 of the shaft 120, such as by cement, a threaded connection, a weld, a braze, or the like. The ball connector 136 is generally hemispherically shaped, particularly on its more proximal portions, and has a distal section 138 which has a smaller outer dimension and is optionally cylindrical in shape. As with the piston body 124, the ball connector 136 is attached to the shaft 120.

With continued reference to FIG. 3, the spring 112 includes a winding 140, advantageously helical; the winding 140 can be a single winding, or can include multiple parallel windings to form the spring 112, and can be wound in either direction. Also, the cross-sectional shape of each winding itself, illustrated in FIG. 3 to be rectangular, can be other shapes, and, when unloaded, the winding 140 can include spaces between the turns of the winding (as illustrated) or be closed. Although helical springs have been illustrated herein other spring shapes may be used including disc or washer springs and solid (e.g., elastomeric or polymeric) springs. The interior of the spring 112 is hollow, and includes a bore 142 which receives portions of the piston 114. The spring arbor 108 includes a cup-shaped proximal portion 144, which includes a blind bore or recess 146 which is open to the inner bore 142 of the spring. The spring 112 is attached to the spring arbor 108 in a known manner, such as by threading a metallic spring onto a metallic spring arbor. A reduced outer diameter shaft 148 extends distally from the cup 144, similar in some respects to shaft 120, and a ball connector 150 is mounted to the shaft 148; the ball connector is advantageously shaped the same as ball connector 136. In the embodiment of FIG. 3, metal particulate wear is reduced by formation of at least the piston body 124 and the ball connectors 136 and 150 from PAEK or other non-metallic material.

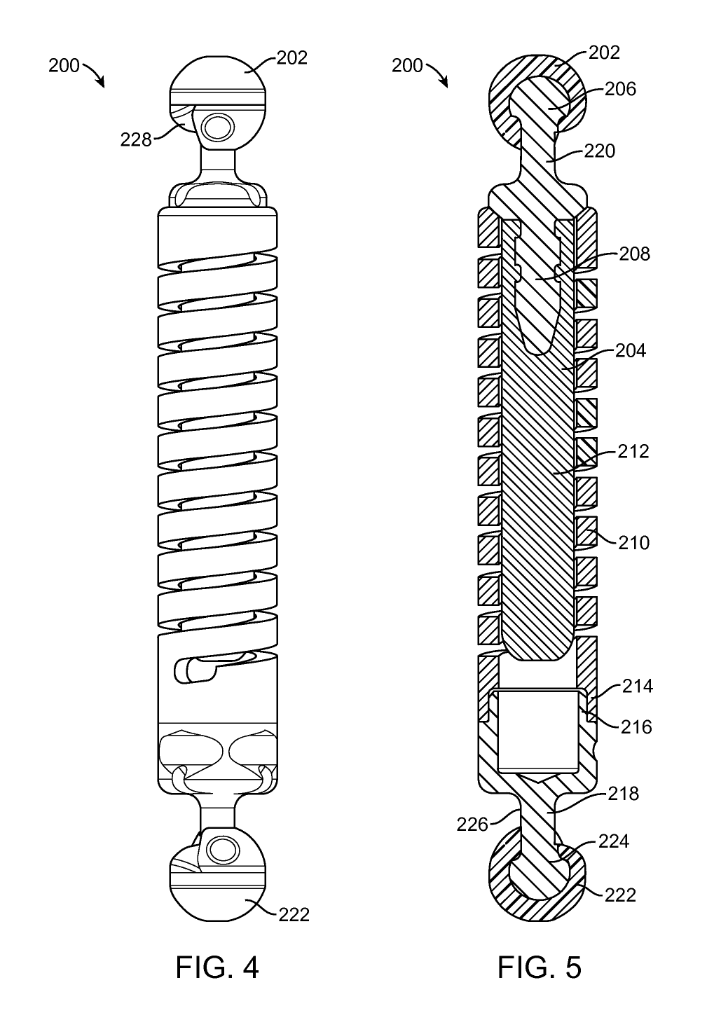

Turning now to FIGS. 4 and 5, another exemplary embodiment of an energy absorbing device 200 is illustrated. The device 200 is similar in some respects to device 106, and includes a proximal ball connector 202. The device includes a spring 210 and a piston 212. A shaft 220 includes an enlarged proximal portion 206, over which the ball connector 202 is overmolded. Similarly, a piston body 204 is molded over a proximal portion 208 of the shaft. The spring 210 includes a distal end 214 which is molded over a reduced diameter portion 216 of a spring arbor 218. The arbor 218 includes a shaft which, like the shaft 220, includes an enlarged distal end 224 over which a ball connector 222 is overmolded. One or both of the ball connectors 202, 222 include a cutout portion 228, better seen in FIG. 4, for inserting the balls into the corresponding sockets in the bases 102, 104.

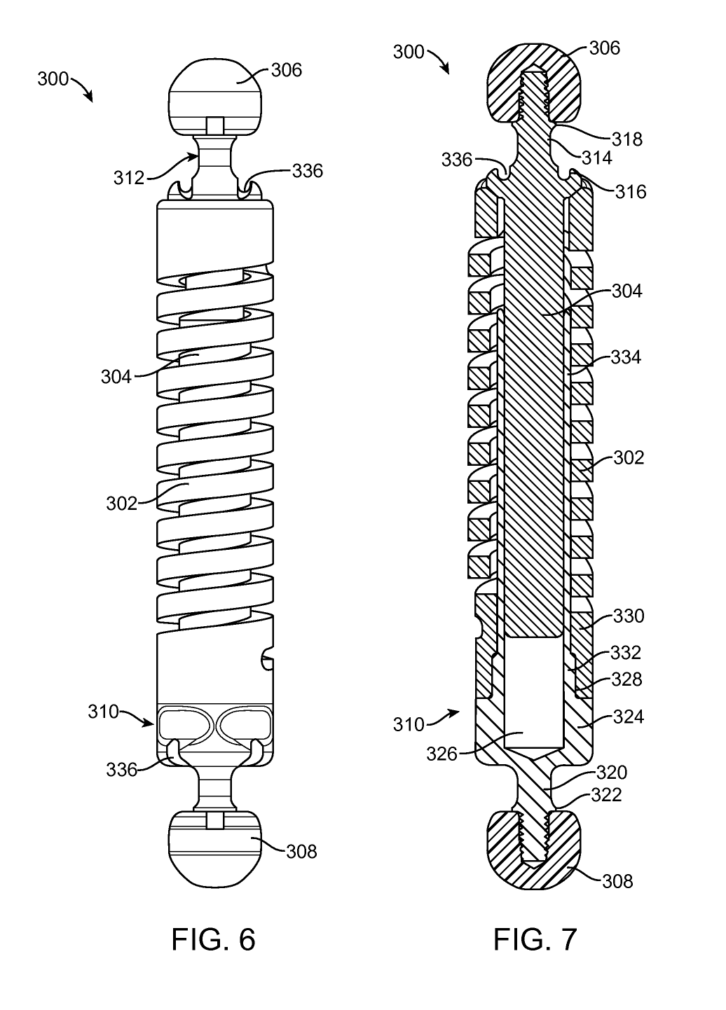

FIGS. 6 and 7 illustrate views of yet another exemplary embodiment of an energy absorbing device 300. Similar to other embodiments described elsewhere herein, the device 300 includes a metallic spring 302 enveloping a non-metallic piston 304. Non-metallic ball connectors 306, 308 are formed on proximal and proximal ends of the device 300, and are connected to a metallic piston base 312 and a metallic spring arbor 310, respectively. In the embodiment 300, some of the non-metallic subcomponents, such as the ball connectors 306, 308 are ultrasonically welded onto the metallic components, which can be advantageous.

Turning now to FIG. 7, the device 300 includes a shaft 314 which includes a radially extending shoulder 316, similar to shoulder 122. The shaft 314 includes a second shoulder 318, proximal of the shoulder 316, which provides a seat or stop for the ultrasonically welded ball connector 306. On the distal end of the device 300, the spring arbor 310 includes a shaft 320 having a distal shoulder 322, similar to shoulder 316. A cup 324 extends proximally from the shaft 320, and includes an interior blind bore 326 and a distally extending, reduced outer diameter cylindrical portion 328. The spring 302 includes a corresponding distal portion 330 which fits over the portion 328, at which an ultrasonic weld 332 is formed. Further optionally, yet advantageously, a hollow, cylindrical piston guide tube 334 extends proximally from the portion 328, and the piston 304 movably extends within the tube 334. Optional recesses or cutouts 336 are formed in the proximally facing portion of the shoulder 122 to receive a constraining cable used to secure the energy absorbing device 300 in a compressed condition during implantation.

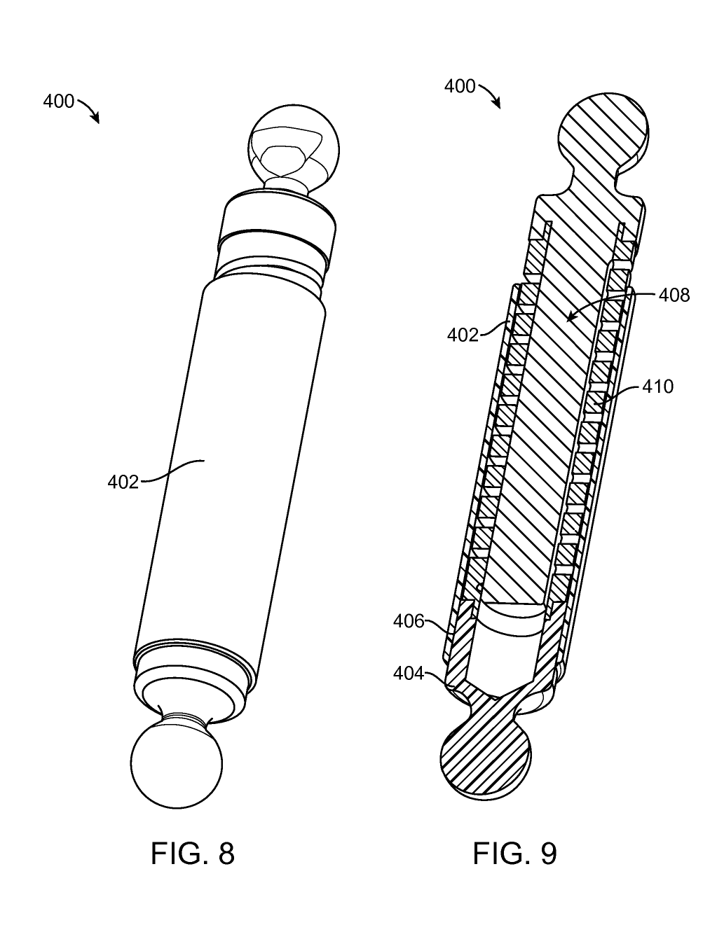

FIGS. 8 and 9 illustrate yet another exemplary embodiment 400 of an energy absorbing device. The device 400 includes an external, tubular sheath 402 which covers at least portions of, and preferably all of, the external surface of a spring 410, so that tissues do not interfere with the functioning of the spring. To avoid any metal-on-metal contact, when metal is used, the sheath is advantageously formed of PAEK. A piston 408 is movably positioned within the spring 410. The sheath 402 is formed integrally with, or attached to, a spring arbor 404, to which the spring is attached. The sheath 402, when attached to the arbor 404, can be welded, threaded, or otherwise attached to the arbor. In this embodiment, the spring 410 can be free floating and not connected to either spring arbor 404 or the piston 408. In addition, the spring arbor 404 and first ball and shaft can be formed of a single piece while the piston 408 can be formed integrally with the second ball and shaft.

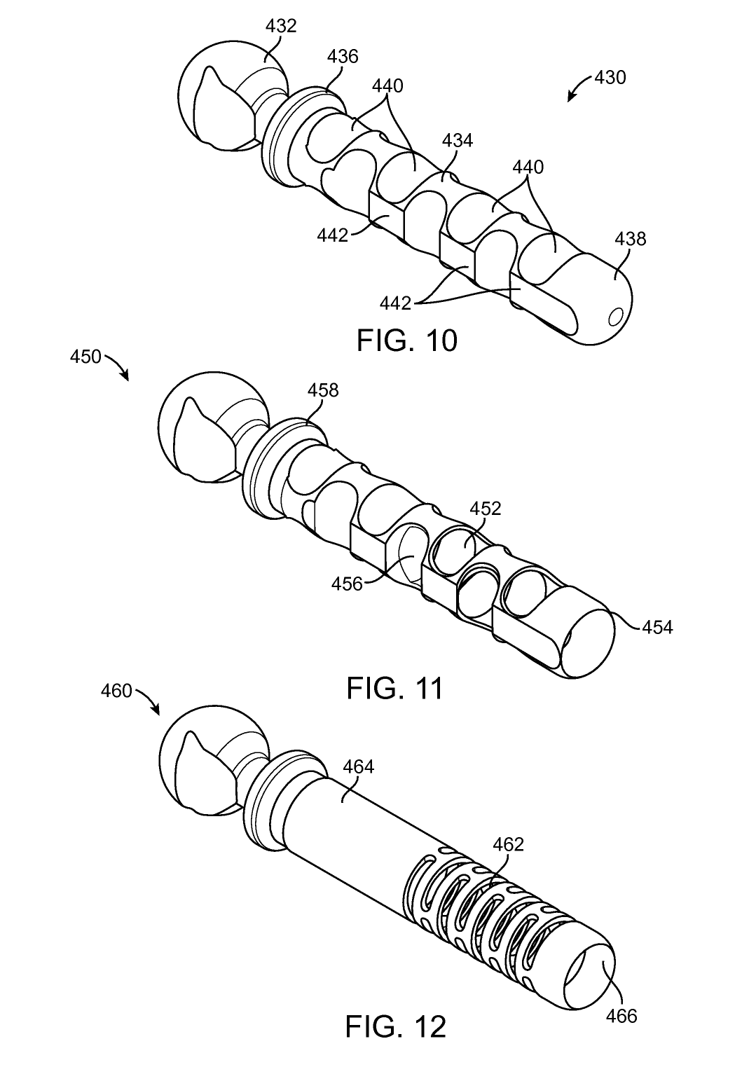

FIG. 10 illustrates a perspective view of yet another exemplary embodiment of a piston 430, one of several exemplary embodiments described herein of pistons which are configured with certain portions that are flexible, and/or to be more flexible in certain directions that in others. The exemplary piston 430 includes a proximal ball connector 432 and a distally extending, generally cylindrical shaft 434, separated by a shoulder 436 which is similar to other shoulders described herein. The shaft 434 includes a rounded distal end 438 opposite the ball connector 432. Along the exterior of the shaft 434, one or more cutouts 440 are formed into the shaft, by which the flexibility of the shaft is changed, and more specifically the shaft is made more flexible where a cutout is formed. The cutouts 440 can be formed at any position along the length of the shaft 434, although forming the cutouts at regular intervals along the entire length of the shaft permits the shaft to be more uniformly flexible along its length. The shape of each cutout 440 can be unique from those of the other cutouts; the cutouts can all be the same shape; or combinations of the same and differently shaped cutouts can be used. In the exemplary embodiment illustrated, each cutout is the same, and forms a part-cylindrical surface in the shaft 434. Further optionally, one or more longitudinally extending flattened sides 442 can alternatively, or in addition to the cutout(s) 440, be formed, which also increases the flexibility of the shaft 434 where present. While only one such flattened side 442 is illustrated, a plurality can be provided, circumferentially arranged on the shaft, in regular or irregular mutual spacings.

The flexible pistons described in FIG. 10 and in the embodiments following provide additional lateral flexibility to the energy absorbing systems. Lateral flexibility of the energy absorber device provided by a combination of a flexible piston and a flexible spring can accommodate side loads that may be applied by the anatomy during articulation of the joint. For example, tissue located between the bones of the joint and the implanted device may apply a side load on the absorber device during flexion. The flexible springs and pistons help to accommodate these side loads. In one example, an arrangement of a flexible piston, such as a solid PAEK piston or a piston having the cutouts described with respect to FIG. 10, in combination with a metallic helical spring, has a flexibility such that a side load of about 5 lbf applied at a midpoint of the piston and spring and transverse the an axis of the piston/spring provides a displacement of about 1 mm. In this example, a side load of about 10 lbf provides a displacement of about 1.7 mm. A spring and piston design providing side displacement of about 0.5-3.0 mm for a side load of 5-10 lbf can be used to accommodate some patient's anatomy which results in tissue impingement.

FIG. 11 illustrates a perspective view of yet another exemplary embodiment of a piston 450 having features which render the piston more flexible than a purely cylindrical piston. The piston 450 includes a longitudinally extending blind bore 452, which extends from the distal end 454 of the piston to a proximal end 456 which is distal of the shoulder 458. As illustrated in FIG. 11, the bore 454 can be combined with other flexibility-imparting features of a piston, as described herein, such as cutouts and flattened sides.

FIG. 12 illustrates a perspective view of yet another exemplary embodiment of a piston 460 having features which render the piston more flexible than a purely cylindrical piston. The piston 460 includes a solid half and a flexible half having part-circumferential slits or slots 462 in the piston body 464, and further optionally a blind bore 466 similar to bore 454; when both the slits or slots 462 and the bore 466 are provided, the slits or slots extend from the exterior surface of the piston body to the bore. The slits or slots 462 can be provided at any longitudinal and/or circumferential position along the piston body 464, to tailor the flexibility of the piston 460 to any desired flexibility profile. When slots 462 are provided, they can have any longitudinal width. With slits, slots, or both, they can be regularly or irregularly positioned on the piston body 464; thus, while the exemplary embodiment of FIG. 12 illustrates slots 462 formed in an alternating and regular pattern, any such pattern can be implemented. In another embodiment, the slits formed in the piston can be in the form of helical slits creating a piston in the form of a helical spring.

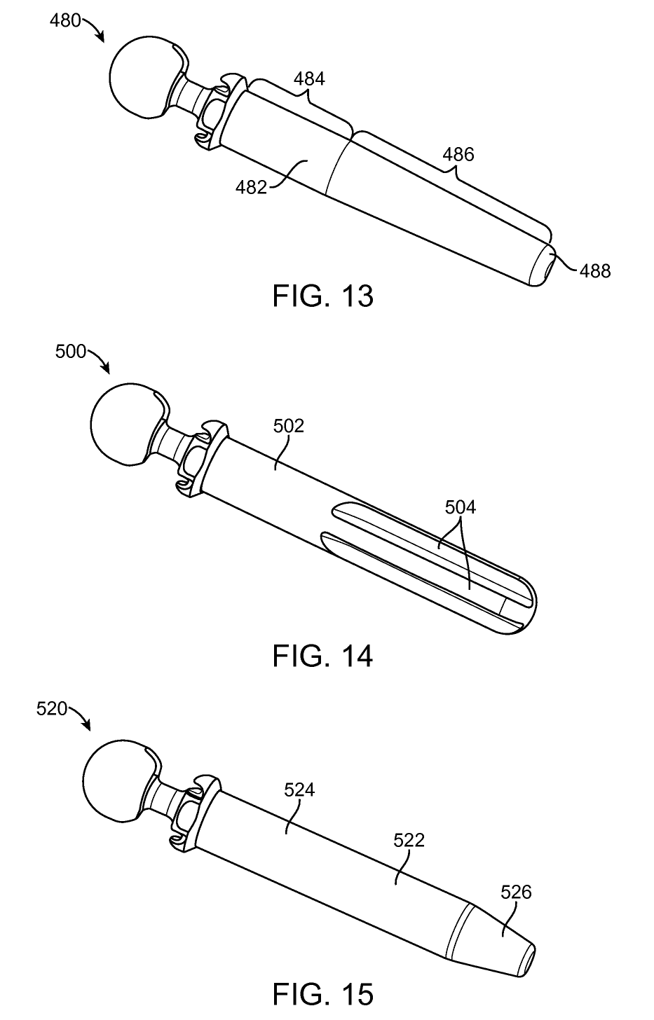

FIG. 13 illustrates yet another perspective view of an exemplary embodiment of a piston 480 having features which render the piston more flexible than a purely cylindrical piston. The piston 480 includes a piston body 482 having a proximal portion 484 which is cylindrical, and a distal portion 486, adjacent to the proximal portion, which is tapered and frustoconical. The distal end of the piston body 382 optionally includes a rounded tip 488 of an outer diameter selected so that the flexibility of the distal portion 486 is as desired.

FIG. 14 illustrates a perspective view of yet another exemplary embodiment of a piston 500 having features which render the piston more flexible than a purely cylindrical piston. The piston 500 includes a piston body 502 which is generally cylindrical, and includes at least one, and advantageously a plurality, of longitudinally extending slots 504. The slots 504, when more than one is provided, are preferably circumferentially evenly distributed in the piston body 502. While it is preferable that each slot, when more than one slot is provided, had the same depth, so that the flexibility of the portion of the piston body 502 including the slots is more circumferentially uniform, other embodiments include slots of different depths. The longitudinal length and position of each slot can also be varied, in accordance with the desire to tailor the flexibility of the piston body; thus, while FIG. 14 illustrates slots 504 extending proximally the same length from the distal end of the piston body 502, other embodiments can include slots of different lengths, originating and terminating at different positions along the piston body, of different widths, cross-sectional profiles, and/or depths.

FIG. 15 illustrates a perspective view of yet another exemplary embodiment of a piston 520 having features which render the piston more flexible than a purely cylindrical piston, which is similar in some respects to piston 480. The piston 520 includes a piston body 522 having a cylindrical section 524 and an adjacent frustoconical section 526 which is significantly shorter than the section 524.

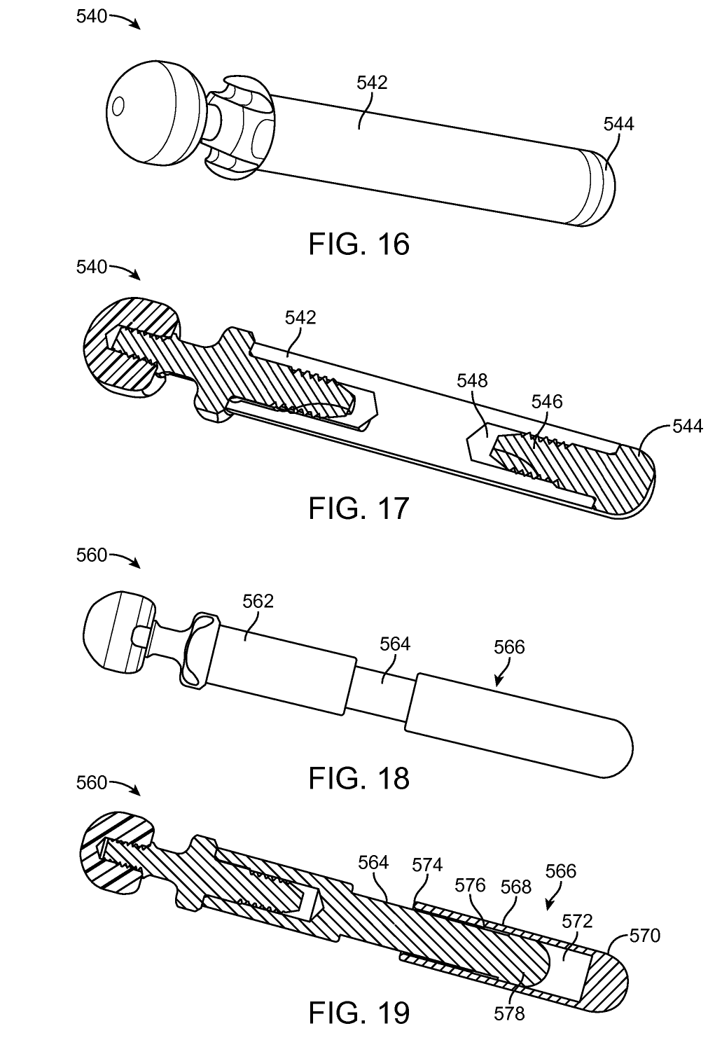

FIG. 16 illustrates a perspective view of yet another exemplary embodiment of a piston 540. The piston 540 includes a piston body 542, which can be the same as any of the piston bodies described herein, and a distal piston end cap 544 formed of a material different from that of the piston body, and more preferably of a biocompatible metal. The provision of a metal end cap can improve the durability of the piston, as the distal end of the piston 540 tends to ride in contact with the interior surface of the spring (not illustrated in FIG. 16) and thus is subject to greater potential wear. FIG. 17 illustrates a longitudinal cross-sectional view of piston 540, showing exemplary constructions of the piston. By way of example only, the piston end cap 544 can include a proximally extending shaft 546 which is held in place in a distally extending blind bore 548, e.g., with a self-tapping screw thread formed on the shaft 546, by insert molding, pressing, over-molding, or any other suitable method.

FIGS. 18 and 19 illustrate perspective and longitudinal cross-sectional views, respectively, of yet another exemplary embodiment of a piston 560. The piston body 562 includes a distal, reduced diameter portion 564 and a telescoping proximal piston extension 566 which freely rides over the exterior of the portion 564 for increased range of travel of the piston without dislocation of the piston from the spring. The extension 566 includes a tubular portion 568 and a proximal end cap 570 at the proximal end of the portion 568, forming a blind bore 570 in the extension 560 leading to a distal opening 574. A lip 576 is formed on the interior surface of the tubular portion 568, which cooperates with a corresponding enlarged proximal end of the body 564, which has a larger outer diameter than the inner diameter of the lip 576, to prevent the tubular portion 566 from sliding off of the potion 564. Otherwise, the extension 566 is free to slide along and rotate relative to the piston body 562, which can reduce the wear between the piston body and a spring positioned around the piston body.

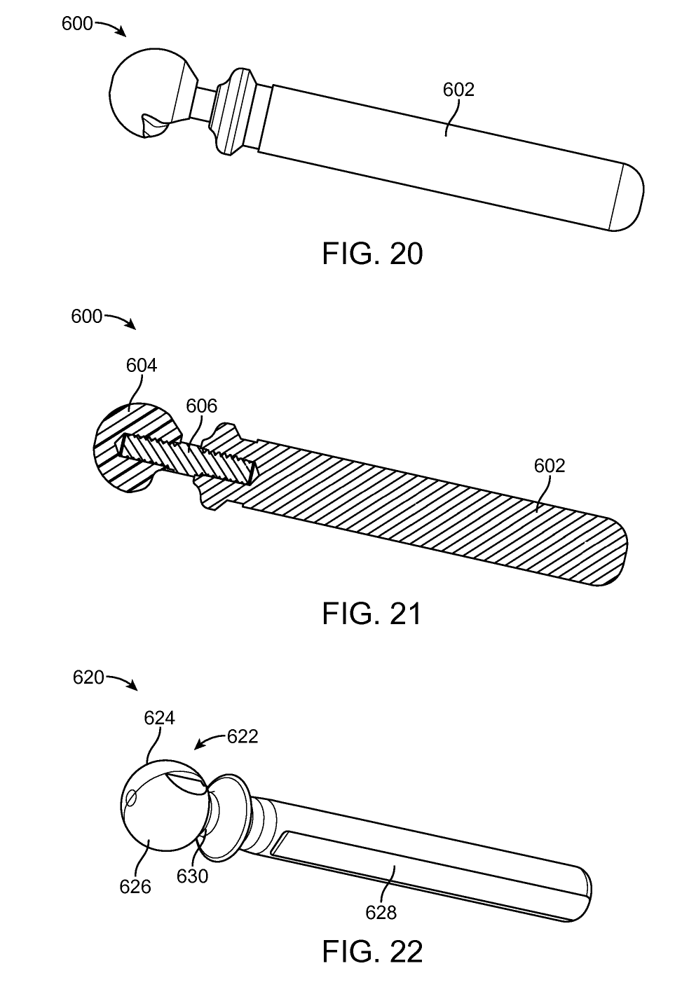

FIGS. 20 and 21 illustrate perspective and cross-sectional, respectively, views of another exemplary embodiment of a piston 600, which includes a unitary, cylindrical piston body 602. The piston 600 includes a ball connector 604, similar to others described herein, a connector shaft 606, and a unitary piston body 602. The connector shaft 606 may be formed of a metallic material for added strength at the narrow neck portion of the piston assembly, while the piston 602 and ball 604 may be formed of PAEK or other non-metallic material for reduced metallic wear and improved MRI compatibility.

FIG. 22 illustrates a perspective view of yet another exemplary embodiment of a piston 620. The piston 620 includes a split ball connector 622, which is formed of two hemispherically shaped portions 624, 626 which are joined together over a connector shaft 630. The piston 620 optionally includes one or more longitudinal flattened sides 628, similar to sides 442, to modify the flexibility of the piston.

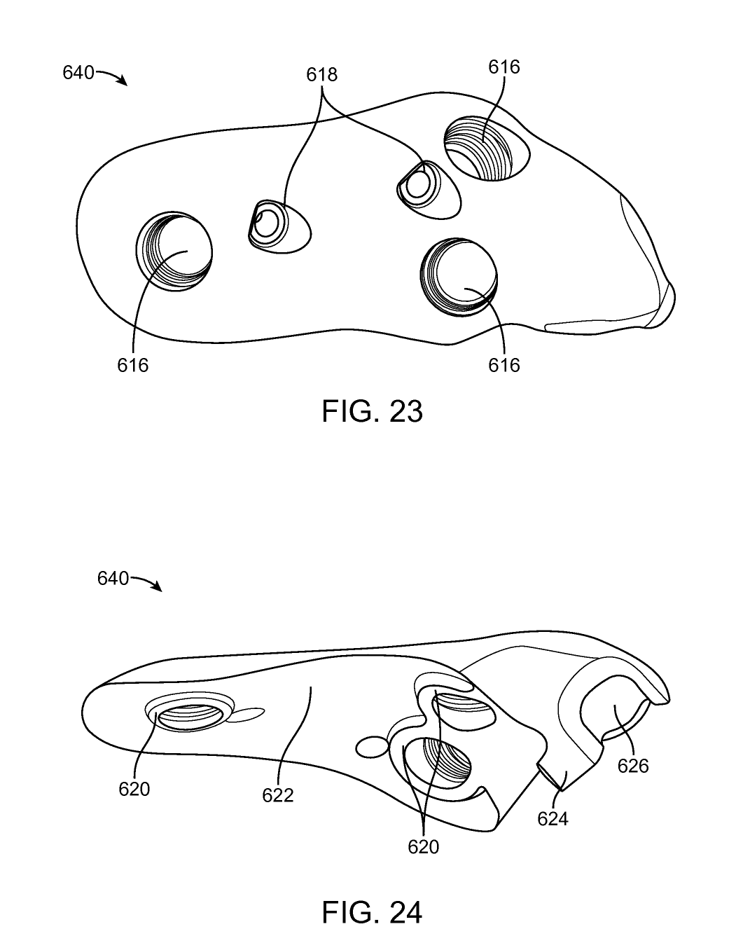

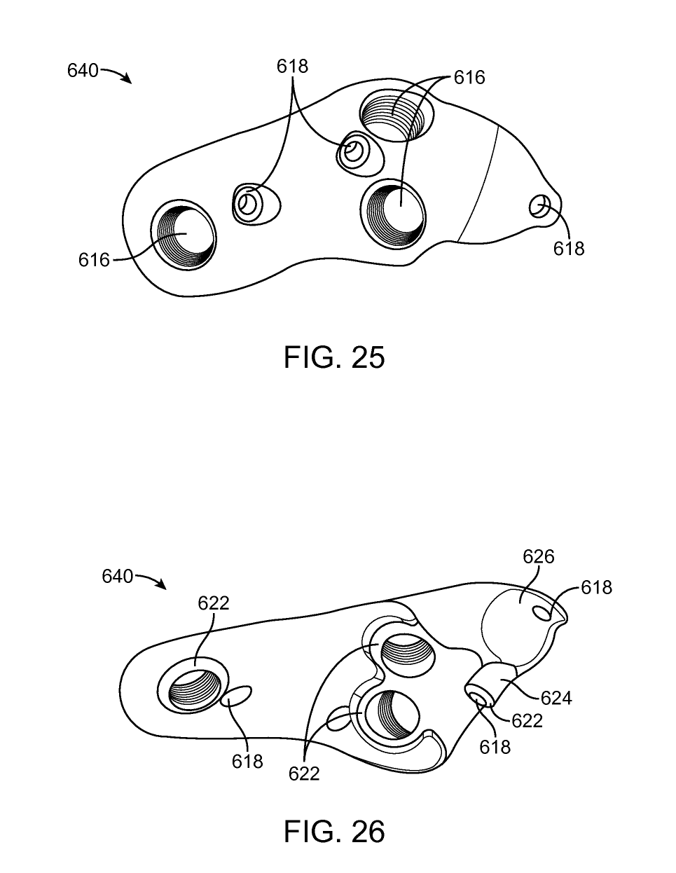

FIGS. 23-26 illustrate top plan and a bottom perspective views of an exemplary femoral base 640. The base 640 includes prethreaded screw holes 616 for receiving correspondingly configured bone screws to fixedly mount the base 640 to the femur of a patient; however, the base can be configured to mount to other bones as well. Alternatively, self-tapping screw holes can be used in place of the prethreaded screw holes 616 when a non-metallic base is used with a harder metallic screw. With reference to FIG. 24, the base 640 includes periostium-contacting rings 620 projecting from the bottom, bone facing surface 622 around the holes 616. The rings 620 assist in positioning the base 640 to the bone and accommodating differing bone contours of different patients by providing three point contact between the base 640 and the bone surface. The rings 620 function as protrusions which provide secure contact with the bone and also allow the bases to be placed on the bone with minimal disturbance of the periosteum, a membrane that lines the outer surface of the bones. Each of the plurality of contacting rings 620 ordinarily has a height of about 2-5 mm, preferably about 3 mm above the main bone facing surface 622 of the base.

The base also advantageously includes a stand-off 624 extending downwardly and away from a socket connector 626, which is configured to receive a ball connector of an energy absorbing device as described elsewhere herein. The stand-off 624 assists in stabilizing the base relative to the bone to which the base 640 is mounted, and to a connected energy absorbing device. The stand-off 624 assists in maintaining a sufficient distance between the ball and socket connection and the underlying tissue to limit impingement of the tissue on the energy absorbing device. The base 640 also may include one or more through holes for temporarily receiving a K-wire or other securing member during positioning and securing of the implant to the bone. The term K-wire as used herein is intended to mean any guide pin, Steinmann pin or Kirschner wire.

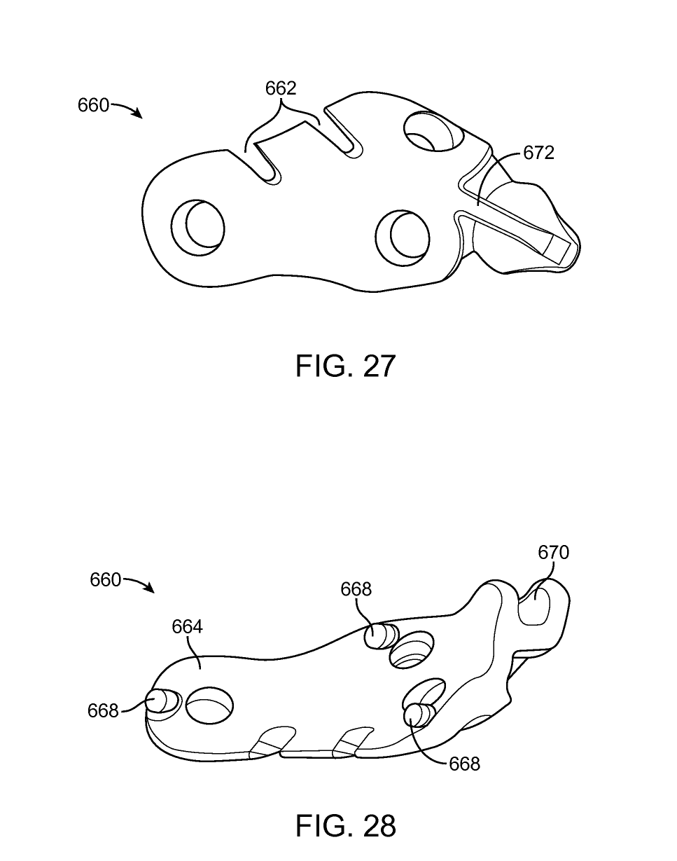

FIGS. 27 and 28 illustrate top and bottom perspective views, respectively, of an exemplary femoral base 660, which has some features which are similar to those of base 640. Different from the base 640, base 660 includes at least one, and preferably at least a pair, of slots 662 which are positioned and sized to receive, at least temporarily, a K-wire in each slot during implantation of the femoral base, as will be described in greater detail below. Additionally, a socket connector 670 is formed in a portion of the base 660 which is connected via a support rib 672, which reduces the amount of material needed to form the base 660 relative to the base 640, while still providing sufficient mechanical support to the socket. The base 660 further includes at least one, and advantageously numerous, compressible, periosteum-engaging feet 668 extending from the bottom surface 664 of the base. The feet 668 are provided to assist in stabilizing the base 660 relative to the bone to which the base is mounted.

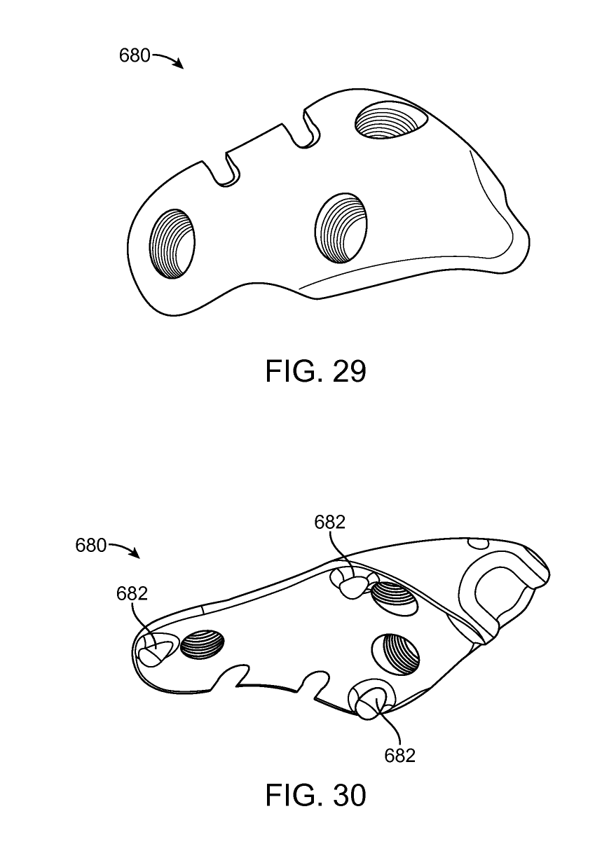

FIGS. 29 and 30 illustrate top and bottom perspective views, respectively, of an exemplary low-contact base 680, which is similar in some respects to bases 102, 104, described elsewhere herein, and to low-contact bases described in co-pending U.S. application Ser. No. 13/488,102, the entirety of which is incorporated by reference herein. Base 680 includes, as illustrated in FIG. 30, foot-shaped stand-offs 682 adjacent to the screw holes, which are provided to help stabilize the base to the bone to which it is mounted. Alternately or in addition, ring-shaped standoffs, such as those shown in the embodiments of FIGS. 23-26 may be provided. The bases described herein in FIGS. 23-30 are formed in a unitary one piece configuration including a first end offset from the bone and having one half of a ball and socket joint (socket portion) and a second end with a contoured bone securing portion and screw holes for securing to the bone.

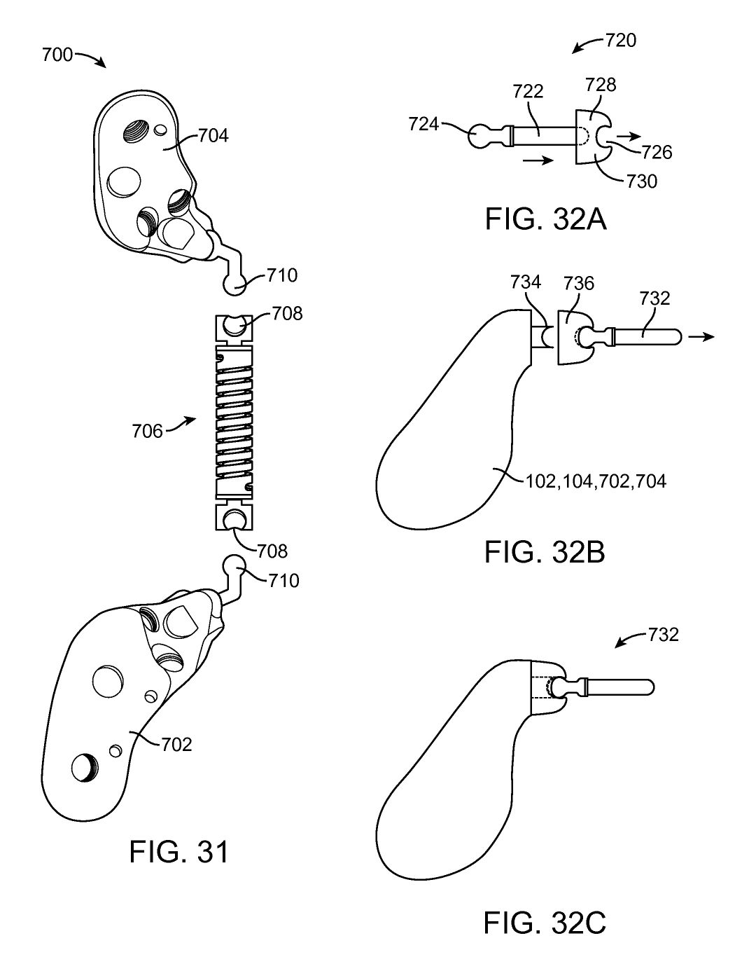

FIG. 31 illustrates an exploded bottom plan view of yet another exemplary energy absorbing system 700, in which the ball and socket connectors are reversed relative to other embodiments described herein. The system 700 includes bases 702, 704, which are configured to be mounted to bones on either side of a joint, e.g., the femur and tibia, and an energy absorbing device 706 which is connected to and extends between both the bases 702 and 704. Each of the bases 702, 704 includes a ball connector 710, similar to other ball connectors described herein, and the energy absorbing device 706 includes a pair of socket connectors 708 on opposite ends thereof, configured to receive and connect with the ball connectors.

FIGS. 32A-32C illustrate details of yet another exemplary connector, 720, which is a captured-ball connector. A piston 722 including a ball connector 724 on one end is inserted into a socket body 728 including a socket connector 726 and a lumen 730 formed therein, with the lumen sized such that the piston 722 will slide through the socket body. By sliding the socket body 728 towards the ball (FIG. 32B), the socket connector 726 receives and retains the ball connector 724. A remaining back side articulation surface of the socket is provided on a mating structure of the base. The base, such as bases 102, 104, 702, 704, includes a mating structure 734, and the subassembly 732 includes a complementary mating structure 736, so that the subassembly 732 can be mounted to the base with the piston 722. Once the base is mounted and secured to the socket body 728, the ball and socket joint is complete and the ball is not removable from the socket (see FIG. 32C).

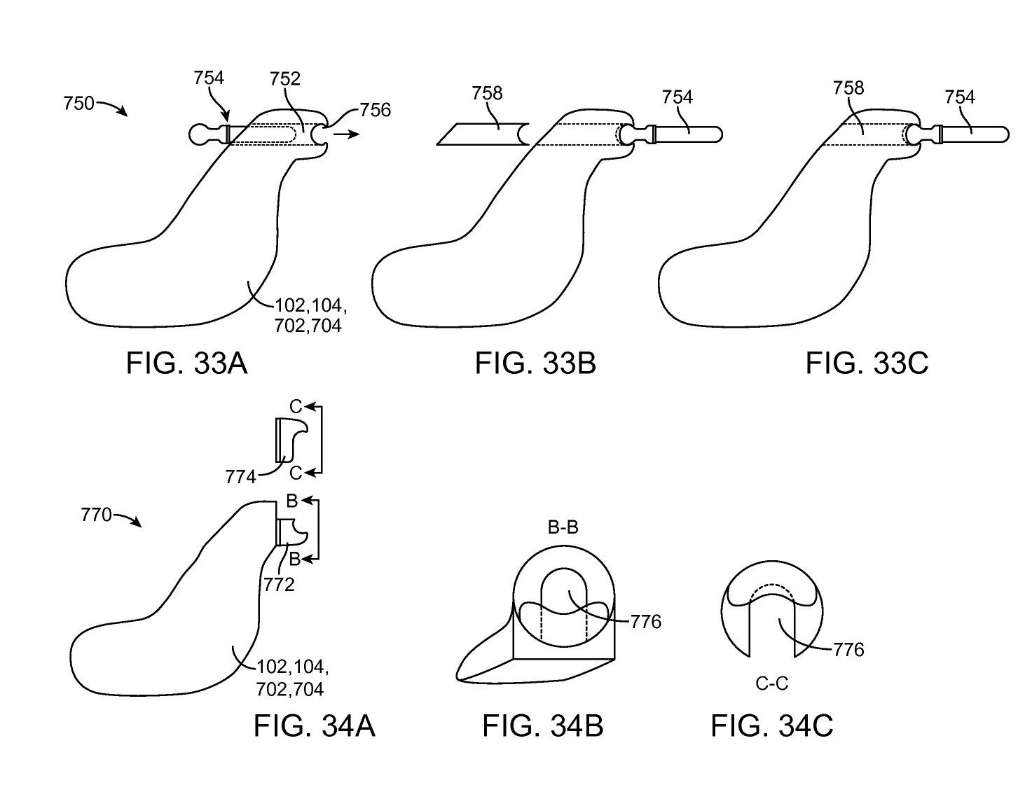

FIGS. 33A-33C illustrate yet another exemplary connector, 750, which is another form of a captured-ball connector in which once the connector is assembled, the ball is no longer removable from the socket. A base, such as bases 102, 104, 702, 704, includes a lumen 752, in which a piston with a ball connector, 754, slidably extends towards a socket connector 756 at and end of the lumen. Once the connector 754 has been inserted completely through the lumen 752, such that the ball is captured in the socket 756 (FIG. 33B), a plug 758 is inserted behind the connector 754 and fixed relative to the base providing a portion of the bearing surface of the socket and preventing the connector 754 from backing out (FIG. 33C).

FIGS. 34A-34C illustrate views (FIGS. 34B and 34C being taken at lines B-B, C-C, respectively, in FIG. 34A) of yet another exemplary connector 770. A base, such as bases 102, 104, 702, 704, includes a portion to which a partial socket connector 772 is integrally formed, or mounted. A complementary partial socket connector 774 includes portions which can be slid over the outer surface of the connector 772, so that together they form a complete socket connector. The ball connector 754 is first mounted into the partial socket connector 772, and the partial socket connector 774 is then slid over both the ball connector 754 and the partial socket connector 772, capturing the ball in the complete socket formed by the two partial socket connectors. While the exemplary embodiment of FIGS. 34A-C illustrate the partial socket connector 774 sliding down onto partial socket connector 772, the two pieces can alternatively be structured to be slid from any direction. While any suitable locking structure can be used, tongue-and-groove type complementary shaped surfaces on the partial socket connectors 772, 774, including dove-tail shapes, can prove advantageous. To hold the two partial socket connectors together when slid together and with a ball connector captured therein, the two partial socket connectors can be held together with one or more pins, set screws, snap-fit connections, welded, or simply glued together. The two partial socket connectors can be formed of any one or more of numerous materials, including metals, polymers (with or without fillers and reinforcing inclusions), and ceramics.

One other example of a manner for engaging a ball and a socket is to provide an internally threaded ball similar to the ball 150 shown in FIGS. 2 and 3. This ball 150 can be dropped into a socket sideways and then the shaft can be threaded into the ball. With this version of a ball and socket, the ball is captured in an integral socket and cannot be removed from the socket without unthreading the ball from the shaft.

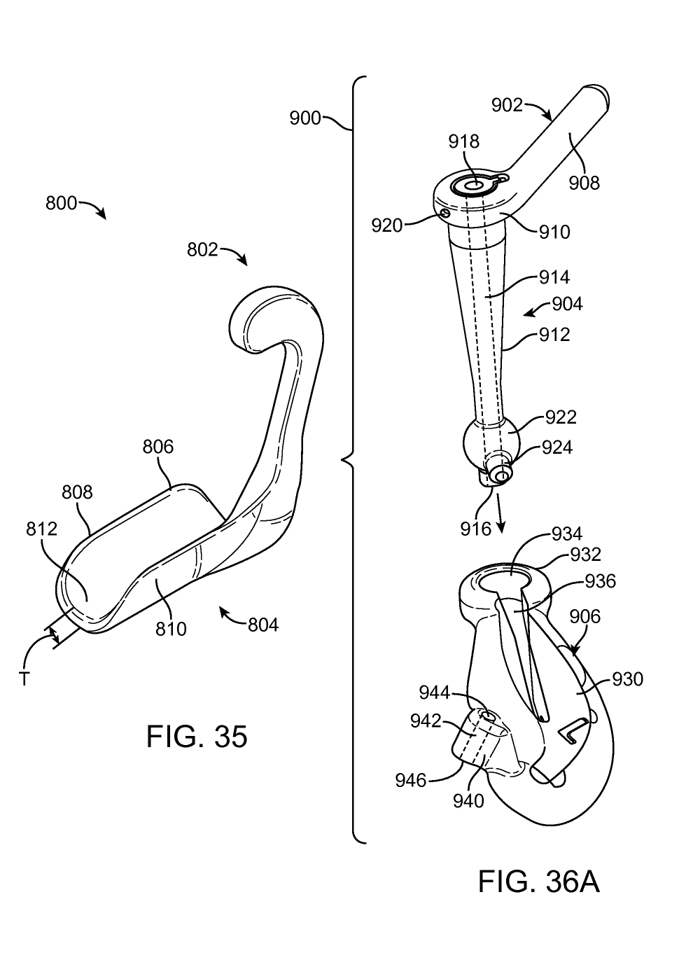

FIG. 35 illustrates a perspective view of an exemplary absorber spacer, 800. As described in greater detail elsewhere herein, an absorber spacer is useful for setting a minimum distance between an energy absorbing device, such as device 106, and underlying tissues when implanted in vivo, to inhibit tissue impingement by the device. With reference to FIG. 35, an exemplary spacer 800 includes a handle 802, which can be J-shaped to assist in its manipulation, and a spacer 804. The spacer 804 is advantageously semi-tubular in shape to match the shape of the energy absorbing device 106, and includes a rounded trough 806 formed from a pair of sidewalls 808, 810, and a bottom 812, leaving an open top. The semi-tubular cylindrical shape may be replaced with other shapes to accommodate energy absorbing devices of different shapes. The thickness T of the bottom 812 is selected so that, when the absorber spacer is positioned with the bottom 812 between an energy absorbing device (e.g., 106) and the patient's underlying tissues, sufficient space is left upon removal of the spacer that the energy absorbing device is unlikely to impinge on those tissues.

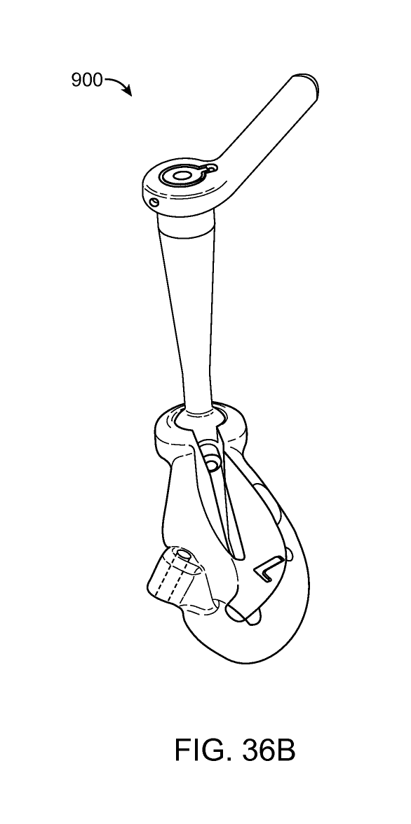

FIGS. 36A and 36B illustrate perspective views of a tibial alignment guide 900 in partially-assembled and fully-assembled configurations, respectively, an exemplary use of which is described elsewhere herein. The guide 900 includes an indicator arm 902, a post 904, and a base 906, which are assembled together when used (see FIG. 36B). The arm 902 includes a straight portion 908, which is useful for determining when the guide is correctly aligned, as described below. A ring-shaped connector 910 is formed at an end of the straight portion 908, and is configured to receive corresponding portions of the post 904 in a predetermined angular orientation. Alternatively, the arm 902 and the post 904 can be integrally formed as a single piece. When formed as separate sub-components, and end of the post 904 is inserted into the ring 910, and the post and ring are secured together, e.g., with a set pin 920.

The post 904 includes an elongate tubular portion 912, including a lumen 914 extending entirely through the portion 912 from a top opening 918 to a bottom opening 916, which is sized to receive a K-wire therethrough. The bottom of the post 904 includes an enlarged bearing 922, from which a locator protrusion 924 laterally extends. While the post 904 is illustrated as being generally tapered, it may be formed of other shapes.

The base 906 includes a base body 930 which is generally shaped similar to a tibial base (e.g., 102). The base 906 includes a toroidal receiver 932 at one end, which is configured to receive the bearing 922 therein and hold the bearing on a downwardly tapered inner surface 934 of the toroid. A slot 936 is formed in the side of the receiver 932 and into the body 930, the portion at the receiver being sized to receive the protrusion 924 and prevent it from moving side-to-side. Thus, when the arm 902 and post 904 are inserted into the base 906, the slot 936 receives the protrusion 924 and accurately orients the straight portion 908 of the indicator arm 902 in a predetermined direction relative to the base 906.

The base 906 also includes a wire receiver 940, formed on a medial side of the base body 930 near an end of the body away from the receiver 932. The wire receiver 940 includes a lumen 942 extending entirely from a top opening 944 to a bottom opening 946, and is sized to receive a K-wire therein, for reasons explained in greater detail elsewhere herein. Alternatively, the receiver 932 and slot 936, and the bearing 922, can be reversed, that is, formed on the other of the post 904 and the base 906.

Exemplary methods of treating a patient with devices as described herein are similar in some respects to those described in the aforementioned co-pending U.S. patent applications. More specifically, once a system, such as system 100, is implanted across a patient's joint, e.g., knee, the system can absorb energy that would otherwise be transmitted through the structures of the joint. When the joint is flexed, the piston 114 slides inside the spring 112, with the proximal end of the piston moving relatively towards the distal end of the spring. This relative distal movement of the piston is unimpeded, because there are no structures of the system 100 which inhibit this motion; however, because the length of the piston is selected to extend most or all of the way along the interior of the spring, and because flexion of the joint is naturally limited, the piston remains inside the spring. When the joint is straightened, or nearly straightened, the shoulder 122 (see FIG. 3) bears against the distalmost portion of the spring, causing the spring to be compressed against its spring force. The compression force, originating in the bones to which the bases 102, 104 are attached, is transmitted through the spring to the spring base or arbor 108, and to the base 102. In this manner, the force, and therefore energy, required to compress the spring bypasses the natural joint across which the system is attached, while exerting no force in the opposite direction once the spring has reached its uncompressed position. The system is designed to absorb either a portion or all of the forces normally transmitted through the natural knee joint. In one example, the energy absorber transmits about twenty to forty pounds of the load normally transmitted through the natural knee joint to provide pain relief to the joint.

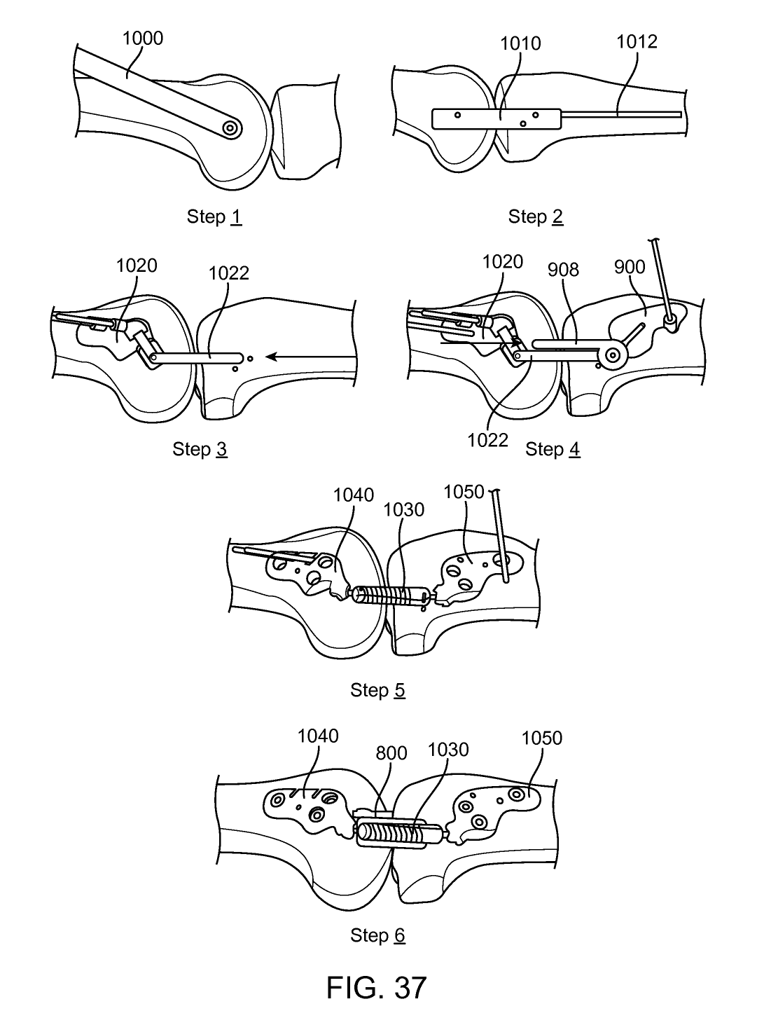

According to an exemplary embodiment, a method of implanting an energy absorbing system, extracapsularly, includes a number of steps in an orthopedic procedure. FIG. 37 illustrates, at a relatively high level of abstraction, steps of an exemplary method. With continued reference to FIG. 37, the following detailed, yet exemplary method steps can be followed.

The surgeon accesses the medial aspect of the operative knee with two 5-8 cm incisions connected by a subcutaneous tissue tunnel. The femoral base is placed sub-vastus on the femur, and the tibial base is placed anterior to the insertion of the pes anserinus. The tibial base placement is on the anterior-medial aspect of the tibia in the following procedure. As shown in FIG. 37, Step 1, a targeting tool 1000 can be placed near the midpoint of Blumensaat's Line to plan the distal edge of the femoral incision. This initial targeting step assists with planning general location of the femoral incision.

The targeting tool 1000 can then be used to place a Kirschner wire (K-wire). When the K-wire appears as a dot within the rings of the targeting tool this confirms that the K-wire is entering the femur at the correct angle and the K-wire can be inserted into the bone for use in positioning the femoral base. With the knee in full extension, a femoral alignment guide 1010 as shown by way of example in U.S. patent application Ser. No. 13/564,095, can be placed over the K-wire shown in Step 2 of FIG. 37. The indicator 1012 of the femoral alignment guide 1010 is aligned so that it is parallel to the tibial axis. A second and third K-wire are then inserted into the tibial K-wire holes of the femoral alignment guide 1010. The distal K-wire represents the location of the tibial ball of the absorber and will assist with femoral base selection and alignment. A femoral base is selected, such as by use of a femoral trial device 1020 or series of femoral trial devices. One example of a femoral trial device is shown in U.S. patent application Ser. No. 13/564,095.

With the femoral trial device 1020 over the target femoral K-wire, an indicator of the femoral trial device is aligned with the distal tibial K-wire placed in Step 2. The indicator 1022 of the femoral trial device represents the correct orientation of the absorber 1030. Two K-wires can be inserted through the holes in the upper portion of the femoral trial 1020 in Step 3 to located the femoral base 1040 in Step 5.

With the femoral trial still in place, Step 4 shows the insertion of the tibial positioning guide 900 (described elsewhere herein) over the distal tibial K-wire. The indicator 908 of the tibial positioning guide 900 is positioned parallel to the femoral trial indicator 1022. The indicator of the tibial positioning guide represents the correct orientation of the absorber 1030 in Steps 5 and 6. A K-wire is then inserted through the hole in the distal portion of the tibial positioning guide 900. This K-wire will guide tibial base orientation during implantation.

The femoral trial 1020 and the tibial positioning guide 900 can then be removed in preparation for insertion of the implant.

In one example, the implant including a femoral base, an absorber, and a tibial base, all connected together; is then inserted through the tibial incision to bring the femoral base 1040 through the tunnel posterior to the femoral K-wires. The femoral base 1040 includes slots which allow the base to be slid onto the two femoral K-wires to position the base. The femoral base 1040 can also be stabilized by inserting a K-wire through the hole in the femoral base.

The absorber spacer 800 (described elsewhere herein) can be inserted in through the femoral or tibial incision and beneath the absorber 1030. The absorber spacer 800 assists in setting a minimum spacing between the absorber and the patient's tissues, so there is less of a possibility of tissue impingement by the absorber.

One or more K-wires may be placed next to the absorber to stabilize the absorber during implantation of the bases. For example, a K-wire may be placed on each side of the distal end of the absorber 1030. As shown in Step 5, the tibial base 1050 can be positioned by placing the distal posterior edge of the base against the distal-most tibial K-wire positioned in Step 4. The posterior border of the tibial base may contact the pes anserinus. The pes may be slightly elevated prior to tibial base fixation. With the tibial base stabilized with K-wires, the femoral and tibial bases are secured to the bone with bone screws. The order of screw insertion and length of screws is at the discretion of the surgeon.

In a final step shown in Step 6, any restraining device which prevents the energy absorbing device of the system from operation, e.g., a restraining cable is cut and removed and the absorber spacer 800 is removed. After confirming operation of the implant throughout full range of flexion/extension and varus/valgus motion, the incision can be closed.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

We claim:

1. An implantable energy absorbing system useful for absorbing energy from a joint of a patient, the system comprising:
a proximal base configured and arranged for implantation adjacent to said joint;
a distal base configured and arranged for implantation adjacent to said joint on a side of said joint opposite said proximal base; and
an energy absorbing device movably disposed between the proximal base and the distal base, the energy absorbing device comprising a spring element having a hollow interior, and a piston at least partially located in said spring element hollow interior, wherein at least one of the spring and the piston is formed of a non-metallic material.

2. A system according to claim 1, wherein the spring is formed of a metallic material and the piston is formed of a non-metallic material.

3. A system according to claim 1, wherein the energy absorbing device and the two bases comprise two ball-and-socket connectors connecting together each base with the energy absorbing device.

4. A system according to claim 3, wherein:
the energy absorbing device comprises proximal and distal ends; and
each of said ball-and-socket connectors comprises a socket on one of said bases and a ball on each of said absorbing device proximal and distal ends, each of said balls being rotatably received in one of said sockets.

5. A system according to claim 1, wherein:
said at least one base comprises a removable socket connector and a mating structure on said base, the mating structure being configured and arranged to receive said removable socket connector;
the removable socket connector includes a socket body, a socket formed in said socket body configured to receive said ball, and a lumen extending from said socket; and
said piston comprises a ball and is sized so that said piston can be pushed through said lumen until said ball is captured in said socket.

6. A system according to claim 5, wherein:
at least one of said bases comprises a lumen having two ends, said socket being located at one of said base lumen ends;
said piston comprises a ball and is sized so that said piston can be pushed through said lumen until said ball is captured in said socket.

7. A system according to claim 6, further comprising a plug sized to at least partially fill said base lumen.

8. A system according to claim 1, wherein said base comprises a socket body split in two portions, each of said portions including a mating portion which permits the two socket body portions to be removably connected together.

9. A system according to claim 1, wherein said spring comprises a helical spring.

10. A system according to claim 1, wherein said spring hollow interior has a length, and said piston extends within said spring hollow interior a distance less than said length.

11. A system according to claim 1, wherein the spring is not secured to opposite ends of the energy absorbing device and is floating on the piston.

12. A system according to claim 1, wherein said energy absorbing device further comprises:
two ends, the piston and spring extending toward each other in opposite directions from each of said ends; and
a piston guide tube extending within said spring and around said piston.

13. A system according to claim 1, wherein said energy absorbing device further comprises:
two ends, the piston and spring extending toward each other in opposite directions from each of said ends; and
a tubular sheath positioned on the outside of at least part of the spring, the sheath being connected to one of said two end.

14. A system according to claim 1, wherein said piston comprises lateral cutouts.

15. A system according to claim 1, wherein said piston comprises:
a base end adjacent one of said bases;
a free end opposite said base end; and
a blind bore extending along said piston from said free end.

16. A system according to claim 15, wherein said piston further comprises:
at least one lateral cutout extending between said blind bore and the exterior of said piston.

17. A system according to claim 16, wherein said at least one lateral cutout comprises at least one part-circumferential cutout.

18. A system according to claim 16, wherein said at least one lateral cutout comprises a plurality of part-circumferential cutouts spaced along said piston between said ends.

19. A system according to claim 1, wherein said piston comprises a frustoconical taper along its length.

20. A system according to claim 1, wherein said piston comprises at least one groove extending in a direction between said ends.

21. A system according to claim 1, wherein said piston comprises:
a base end adjacent one of said bases;
a free end opposite said base end;
wherein said free end comprises a metal tip; and
wherein portions of said piston between said metal tip and said base end are formed of PAEK.

22. A system according to claim 1, wherein said piston comprises:
a base end adjacent one of said bases;
a free end opposite said base end; and
a telescoping piston extension positioned around said free end.

23. A system according to claim 22, wherein said telescoping piston extension includes a blind bore having an inner diameter, and said piston free end has an outer diameter less than said blind bore inner diameter such that said telescoping piston extension can slide along said piston free end.

24. A system according to claim 23, wherein said telescoping piston extension includes a lip on said blind bore, and said piston includes a lip adjacent said free end sized to catch on said telescoping piston extension lip and prevent said telescoping piston extension from sliding off said piston free end.

25. A system according to claim 1, wherein said piston comprises at least one flattened exterior portion.

26. A system according to claim 1, wherein at least one of said bases comprises:
a top surface and a periosteum-contacting surface;
at least one screw hole extending through said base between said top surface and said bone-contacting surface; and
at least one periosteum-contacting ring on and extending outward from said periosteum-contacting surface, adjacent to and surrounding said at least one screw hole.

27. A system according to claim 1, wherein at least one of said bases comprises:
a top surface and a periosteum-contacting surface; and
at least one standoff extending from said periosteum-contacting surface.

28. A system according to claim 1, wherein at least one of said bases comprises slots formed through the base.

29. A system according to claim 1, wherein the piston is formed of unfilled or reinforced PAEK.

30. An implantable energy absorbing system useful for absorbing energy from a joint of a patient, the system comprising:
a proximal base configured and arranged for implantation adjacent to said joint;
a distal base configured and arranged for implantation adjacent to said joint on a side of said joint opposite said proximal base; and
an energy absorbing device attached to both the proximal base and the distal base, the energy absorbing device comprising a spring having a hollow interior, and a piston at least partially located in said spring hollow interior;
wherein the energy absorbing device and the two bases comprise two ball-and-socket connectors connecting together each base with the energy absorbing device, at least one of the ball and socket is non-metallic.

31. A base useful for implantation adjacent to a joint, the base comprising:
a top surface and a periosteum-contacting surface;
at least one screw hole extending through said base between said top surface and said bone-contacting surface; and
at least one periosteum-contacting ring on and extending outward from said periosteum-contacting surface, adjacent to and surrounding said at least one screw hole, wherein the base is a one piece base formed of a non-metallic material.

32. A base according to claim 31, wherein said non-metallic material is PEAK.

33. An energy absorber spacer comprising:
a trough-shaped portion having two sidewalls, a bottom wall connected to each of the sidewalls, and an open top; and
a handle extending from said trough-shaped portion and away from said bottom wall.

34. A system according to claim 33, wherein said bottom wall and said two sidewalls define a trough with open ends.

35. A tibial alignment guide comprising:
a base having base body and a pin receiver on said base body including a through lumen; and
a post having a through lumen and first and second ends; and
an indicator arm;
wherein the post and the indicator arm comprise complementary mating structures which permit the indicator arm to be mounted adjacent to said post first end extending away from the post at a single fixed angular orientation; and
wherein said post second end and said base together comprise a removable connector configured and arranged to permit the post to be connected to the base at a single, fixed angular orientation.

36. A system according to claim 35, wherein said removable connector comprises:
an enlarged bearing and a protrusion extending laterally from said bearing;
a toroidal receiver including an interior surface sized and configured to receive said enlarged bearing; and
a slot in said toroidal receiver sized to receive said protrusion, such that when said enlarged bearing is positioned in said toroidal receiver with said protrusion located in said tapered slot, said indicator arm is oriented in a single fixed angular orientation relative to said post lumen and said base pin receiver.

37. A method of implanting an energy absorbing system into a patient having a joint with a joint line, the method comprising:
forming a femoral incision that begins slightly distal to a midpoint of Blumensaat's line and extends away from the joint line;
forming a tibial incision that begins at a posterior tibial K-wire hole and extends through the location of the distal tibial K-wire hole;
forming a tissue tunnel from the tibial incision to the femoral incision;
aligning a tibial positioning guide with a femoral trial indicator to correctly align the tibia and femur;
inserting a K-wire through a hole in a distal portion of the tibial positioning guide;
positioning two femoral and two tibial K-wires in the femur and tibia, respectively;
removing the femoral trial and the tibial positioning guide; and
inserting a femoral end of an energy absorbing system, including a femoral base, an absorber, and a tibial base, connected together, through the tibial incision and bringing the femoral base through the tunnel posterior to the femoral K-wires.

38. A method according to claim 37, further comprising:
engaging the femoral Base with the two femoral K-wires and stabilizing the femoral base by inserting a K-wire through a hole in the femoral base.

39. A method according to claim 38, further comprising:
setting a minimum spacing between the absorber and the patient's tissues, including introducing an absorber spacer through the femoral incision and beneath the absorber.

40. A method according to claim 39, further comprising:
positioning the absorber parallel to the tibial shaft.

41. A method according to claim 40, further comprising:
mounting the femoral base to the femur; and
mounting the tibial base to the tibia.

42. A method according to claim 41, further comprising:
activating the absorber.

43. A method of implanting an energy absorbing system in a patient, the method comprising:
inserting, as a single interconnected unit, a femoral base, a tibial base, and an absorber rotatably connected to the femoral and tibial bases;
positioning the femoral base on the distal end of a femur;
positioning the tibal base on the proximal end of the tibia;
positioning the absorber across the knee joint to absorb load ordinarily carried by the knee joint; and
securing the femoral and tibial bases to the bone with bone screws.

44. A method according to claim 43, wherein said inserting includes inserting through a tissue tunnel from an incision adjacent the tibia.

45. A method according to claim 43, wherein said positioning the absorber includes positioning the absorber substantially parallel to the tibial axis when the knee joint is at full extension.
